(19) United States
(12) Patent Application Publication
(10) Pub. No.: US 2005/0107703 A1
(43) Pub. Date: May 19, 2005

(54) Ultrasonic imaging with spot focused waves

(76) Inventor: James K. Bullis, Sunnyvale, CA (US)

Correspondence Address: James K. Bullis, 1155 Pimento Ave., Sunnyvale, CA, 94087, US

(21) Appl. No.: 10/667,547

(22) Filed: Sep. 22, 2003

(51) Int. Cl. A61B 8/00

(52) U.S. Cl. 600/442

(57) ABSTRACT

The invention is a system that was developed for medical imaging, with particular attention to breast imaging applications. Spot focused architecture enables very high resolution imaging using very large aperture transducer arrays, where depth of field and focal length cause a small focused spot at a pre-set depth for each transmit-receive operation. Scanning depends on rapid movement of the focus spot throughout an intended object space, so transmit-receive events are overlapped in time. Coded signals are used to suppress interference caused by such overlap. Selection of codes by pre-set correlation is simple, where a correlator channel produces a single image data sample for each spot. Coded signals are compensated for frequency dependent attenuation by the medium to enable wide bandwidth effects. Attenuation leveling and fixed paths to spots enable prediction and compensation for frequency dependent attenuation to enable broad band effects. The architecture provides for bistatic operation of sparse arrays, with hybrid electronic beamformers. It also uses mechanical scanning. Transducer elements are constructed by cutting strips from thin cards of piezo-electric material.

This patent document contains material that is subject to copyright protection. Facsimile reproduction is allowed of the patent document or the patent disclosure as is appears in the Patent and Trademark Office patent file or records as allowed by US patent law, but otherwise all copright rights are reserved.

### CROSS REFERENCE TO RELATED APPLICATIONS

Application Ser. No. 09/975,033 Bullis, filed Oct. 10, 2001, Enhanced Focusing of Propagating Waves By Compensation for Medium Attenuation; application Ser. No. 10/060,591, filed Jan. 30, 2002 Bullis, Channeled Wavefield Transformer

### BACKGROUND OF THE INVENTION

This invention relates to sensing using propagating wave signals. The field of the invention involves propagating waves and transducer operation relative to such waves. The primary area of interest is transmitting waves and receiving waves that arise as a result of objects in a field of view that is a three dimensional volume. Example applications in the field of the invention include ultrasonic medical imaging and ultrasonic industrial inspection.

The background of the invention is technology of devices such as radar and sonar that have evolved from origins in the optical lens and the ocean depth sounder. Terminology used in this disclosure is variously extracted from terminology used in connection with such devices.

The present invention came about as a result of development project that addresses a critical need for an imaging device that will enable examination of patients to determine with confidence that disease is present or absent. A useful device must be safe, economical, and convenient to operate in a clinical environment. While available ultrasound apparatus fills these requirements, image quality is poor due to difficulties that arise with examination of living soft tissue that exists as a volume. In this volume, disease processes are variations of the soft tissue that may occur throughout that volume. There are also variations of healthy tissue that generally occur throughout that volume. Either type of variation gives rise to signals that are sensed. Additionally, in the tissue that contains variations that are being sensed, the same variations cause perturbations of wave propagation. Previous imaging technology has not adequately met the challenge of imaging in this volumetric distribution of tissue variations.

Optical lenses have long been used to focus waves. They either concentrate energy about a point or enable selective sensing of energy from a region about a point.

For either function there is an focusing effect in a region about a focus center point. The size of this region, in a plane perpendicular to the wave propagation direction, is specified as resolution. A method for quantifying the resolution effect was defined by Rayleigh with respect to focusing effects of optical lenses. The Rayleigh Criterion specified the separation required between two objects that would enable them to be perceived as two objects rather than blurred into one. The approximate rule is that angular resolution for a circular aperture is approximately equal to 1.22 times the wavelength divided by the aperture. The aperture is approximately the diameter. This terminology has been adapted to apply to any device that provides a focusing effect, where such devices include telescopes, cameras, and microscopes or radar, sonar, seismic, or medical ultrasound devices. The Rayleigh Criterion provides fundamental performance limits of many types of devices, where such limits are a result of focusing effects. In addition to focusing, the lens implements an imaging process where a field of view is instantaneously scanned to show reflecting objects distributed along an image plane. Well known terminology uses “f number” to indicate degree of focusing, where f is equal to focal length divided by aperture. In most cases, the aperture is approximately the active lens diameter and focal length is the distance from the lens to the center of the focused region. This terminology carries over to other fields that focus waves. In the present document, focusing is described as a number that is equal to f Multiple f values may apply, where the f values are with respect to aperture dimensions that correspond to respective focusing dimensions.

The simple optical microscope is constructed such that samples are laid out on a flat slide, and focusing is effective only for points very close to the plane of the slide. The lens serves both for focusing and instantaneous scanning. This is a form of spot focus device since the depth of field is very small. It is well known in optics that small f numbers indicate small depth of field as well as small increments in lateral resolution.

Foster et al. Transmission of Ultrasound Beams Through Human Tissue—Focusing and Attenuation Studies, Ultrasound In Medicine and Biology, Vol. 5, pp 257-268, Pergamon Press, Ltd., 1979, demonstrated one way focusing in breast tissue. Subsequently, Foster et al, The Ultrasound Macroscope: Initial Studies of Breast Tissue, Ultrasonic Imaging 6, pp243-261, 1984, demonstrated a device that was similar in form to the optical microscope except that it utilized ultrasonic waves instead of light. In this apparatus, thin slices of excised tissue were arranged on slides, to be imaged by a focused transducer. This transducer provided a large aperture effect like the lens does in the optical microscope, though two way focusing was accomplished with the single, focused transducer that enabled both the transmitting effect and the receiving effect. The large aperture effects were fully two dimensional to cause focus in a spot. Scanning the spot in the plane of the slide was accomplished by mechanically moving the transducer along two axes. This device produced pictures in which details of invasive cancer were clearly visible. An obvious adaptation of this microscopic form of device to imaging in living tissue volumes using the demonstrated mechanical scanning methods would entail an examination time that would be unsuitable for examination of a living patient. An additional concern was that propagation in living tissue was not meaningfully represented by a test where the focused waves propagated between transducers and the slide through clear coupling fluid such that there was no disturbance by natural tissue effects.

Another demonstration by Foster et al., Breast Imaging With a Conical Transducer/Annular Array Hybrid Scanner, Ultrasound in Medicine and Biology, Vol. 9, No. 2, pp 151-164, Pergamon Press, Ltd., 1983, involved a novel conical transducer and a wide focused transducer in a device that was tested on living subjects. The conical transducer accomplished focusing along a line that extended in the depth direction into the subject. The aperture was an annular ring that increased in diameter proportionally to increase in depth, which was nearly an ideal effect for uniform resolution of a subject. Effective pulse travel along the line occurred at a speed that was modified by the conical effect. Unfortunately, picture quality did not approach that of the microscope form device, probably due to uneven attenuation effects. Neither did this device compete effectively with operating convenience and motion picture features of the approach that was then coming into general use by the ultrasound industry.

However, development of present day ultrasound technology was more closely related to the ocean depth sounder. This simple device has long been used to sense reflections, where possible bottom depths are scanned using time of arrival of reflection signals. A pulse is transmitted and an echo off the ocean bottom is received, where time of arrival of the echo is measured. A depth resolution rule, similar to the angular resolution rule for lenses, is that depth resolution is approximately half the pulse width multiplied by wave propagation speed.

Devices that sense by transmitting signals and receiving reflected signals are called active systems. Most such active systems operate using the depth sounder form of time based scanning in combination with lens emulating effects that scan in lateral dimensions. This enables sensing in a volumetric field of view that is a volume of space. Such devices include most radar, sonar, ultrasound, and seismic systems. Focusing effects of lenses is emulated by transducer arrays where it is appropriate in the system design. Transmitted waves are directed along lines and the lines are variously steered in direction or shifted in position. While focusing processes are variously used, time based operation in the range dimension is almost always present and it a common denominator in the architecture of most active sensing systems.

Much of the terminology in the ultrasound field has evolved from radar technology. For purposes of this discussion, this terminology needs to be clarified. For radar, the counterpart of depth in ultrasound is usually range. Range is a term that is sometimes used for any device where propagation distance is involved. Transducers and antennas carry out similar system functions. Other radar terminology relates to focusing effects. Focusing provides cross range resolution in a horizontal direction that is also called azimuth resolution. It may also provide cross range resolution in a vertical direction that is also called elevation resolution. Either cross range direction is a lateral direction. Any dimension measured in any lateral direction is a lateral dimension. Focusing takes place in an operation where wave energy is concentrated along a line or where wave energy from a region along a line is selectively sensed. For transducer arrays, such focusing is usually called beamforming, where such terminology refers to an analogy with a light beam, though beamforming refers to both transmitting and receiving operations. Beamforming is a combines signal processing with shaping of transducer arrays to form an active aperture.

For transducer array configurations that are long and narrow, terminology is based on a presumption that the transducer device is laid out, like most radar antennas, with its long dimension horizontal, whether or not this is actually the case. The active transducer device is often rectangular and the longer dimension is in a first lateral direction and this longer dimension determines cross range resolution at the focus region in a cross range direction that is parallel to the first lateral direction. The shorter dimension of the transducer is in a second lateral direction that is perpendicular to the first, and this shorter dimension similarly determines cross range resolution at the focus region in a direction that is parallel to the second lateral direction. Such resolution effects are a result of diffraction effects. For either case, resolution is equal to wavelength times focal length divided by the respective aperture dimension. Adapting the terminology of optics, there is a parameter f for either cross range case where f is equal to focal length divided by the respective aperture.

Radar architecture uses time of arrival to scan in range and the azimuth resolution of beams along with rotation, usually mechanical, to scan in azimuth angle. The radar scan format is thus constructed in dimensions of range versus azimuth angle and the corresponding display format is also range versus azimuth angle. Far field conditions usually apply such that depth of field for azimuth focusing is not critical. The elevation beamwidth is designed to be as wide in angle as necessary to accommodate possible target arrival angles. There is no discrimination capability in elevation provided by that elevation beamwidth. Since there is no focusing in elevation, depth of field due to such focusing is not an issue. A B-scan display was a rectangular display that showed azimuth angle in the horizontal, x-axis direction and range in the vertical, y-axis direction.

The radar form of architecture has been widely adapted to medical ultrasound use where depths of respective tissue variations are indicated by time of echo signal arrival and a cross range position of tissue variations is indicated using lateral position resolving methods. More precisely, it is not depth, but rather distance along the direction of propagation from the transducer to the tissue effect that is being sensed. Lateral resolving methods include both steering beams in azimuth angle and lateral shifting of beams to sense along different beam direction lines. In the context of either lateral position resolving method, common terminology used in the ultrasound industry refers to “firing a line” as a transmit-receive event that results in sensing along respective beam direction lines. While this architectural adaptation has resulted in useful ultrasound equipment, human tissue often presents difficulties that cause unsatisfactory results.

Adapting basic radar architecture to medical ultrasound applications is particularly problematic because of the volumetric distribution of objects. Consequently, The elevation beamwidth is a significant problem. The expected object distribution is such that there are many reflections from along any azimuth and elevation oriented line. Elevation beamwidth then causes sensing of an extensive number of objects that are vertically distributed. Since vertically distributed objects are at the same range, range resolution does not provide a capability to distinguish among them. This failure to resolve means that small detail of disease processes is not discernable. It also means that disease processes that produce weak reflection signals can not be seen because the wide beam does not enable exclusive sensing such that weak signals are buried in a summed signal, where the summed signal includes all tissue effects in the beam as a single image sample. The offending elevation beamwidth is commonly referred to as “slice thickness”.

In a time based system architecture, such as typical ultrasound architecture, it is not possible to provide adequate control of this elevation beamwidth because there is conflict between focusing resolution and the range extent over which range can be scanned using the time base. This becomes especially critical where smaller f number causes a smaller extent in range over which focusing is effective. This depth extent is called depth of field in a manner consistent with optical terminology. When depth of field becomes very small, the result of a given transmit-receive event is a focused range extent that is very short, and the fired line is so short that a useful image can not be formed.

Although elevation beamwidth is difficult to control, azimuth beamwidth is more manageable in radar-like architecture. Because transducer arrays have elements that are laid out along a horizontal line, large horizontal apertures can be implemented to cause narrow azimuth focus. Receiving systems can dynamically vary depth of a focus point along a propagation line to achieve continuously varying focus. Transmit focus is more difficult, since a given transmission must be focused in advance.

Electronic beamforming devices are known in prior art. It is a well known part of radar and sonar technology that has been readily adapted in medical ultrasound devices. An early U.S. Pat. No. 3,039,094 (January 1962) Anderson described a method later called DIMUS for digital, multi-beam steering. The multi-beam steering means that it could receive simultaneously in multiple directions. It was a significant improvement over radar methods, that primarily used phase shifting methods to steer beams, because it used time shifting methods. This meant that wide percentage bandwidth signals could be handled. This was particularly important in sonar. This invention specifies the general signal processing method for the benefit of all these fields. It did not provide a method of simultaneous transmission of waves in different directions.

U.S. Pat. No. 5,598,206 (January 1997) Bullis discussed coding to enable simultaneous transmit focus points, both at different angles and different ranges.

U.S. Pat. No. 5,598,206 (January 1997) Bullis was a device to scan a volume and achieve very good resolution in elevation as well as in azimuth. Focusing in these dimensions was accomplished by interaction between a transmit array and a receive array. The configuration enabled a vertical aperture that was equal to the horizontal aperture. Range was eliminated from the display to make a presentation having a format that was similar to that of the human eye. Although the preferred embodiment lacked range discrimination, range was an important consideration in design of this apparatus, which was configured to take advantage of a distribution of objects that would be mostly along a surface, such as the ocean bottom surface. In the illustrated embodiment, the ocean bottom was the representative surface. Here there would be only a single object along any given radial line that was directed in azimuth and elevation from the sensing device. Because the distance to any such singular object was not known in advance it was necessary to allow for a range extent for possible objects, even though there was not a capability for precise resolution in the range dimension. This led to coding of overlapping transmitted waveforms that would focus over different range segments along a given radial line.

This disclosure also described methods of waveform coding to enable fast scanning over multiple, shortened depth of field range zones as well as multiple, beam steering angles. This invention developed overlapping transmit-receive events to the point of simultaneous operation through linear systems. It included methods of waveform coding with replica codes that were subjected to correlation signal processing.

In typical time based scanning apparatus, such replica correlation becomes complicated, since it must perform a time compression process. Replica correlation is similar to convolution and it involves the same computational processes that require very high rate signal processing. Earlier versions of replica correlation used circulating delay lines to implement this high rate computation. An example is the invention U.S. Pat. No. 2,958,039 (October 1960) Anderson that discussed correlation signal processing, where delay line methods utilized simple time samples in a time compression process to produce a correlation function. This came to be called a DELTIC correlator. These were generally used to select a coded signal from background signals of many types, including signals having other codes. More recently, Fast Fourier transform (FFT) processing became a popular way to implement the replica correlation process in digital computers, though it is still a computationally intensive operation. It was convenient to use in phase and quadrature sampling, often called I and Q sampling, to obtain sample pairs that could be handled as complex numbers in the FFT computation. After an FFT of the received signal, a multiplication of that result with an FFT of a code replica, and an inverse FFT of that product, a complex number form of correlation function was available in the time domain. The square of the complex number function was the correlation power function.

Either the DELTIC method or the FFT method produce a full correlation function that is a time compressed version of a long duration coded waveform. This time compressed version is a form of time based signal that is used to scan in the range dimension in most systems where this methodology is used.

Presentation format is important for effective clinical operation. The most natural format is one where viewing of an image is similar to viewing a scene directly with the human eye. The scanning and resolving arrangement of the previously mentioned invention in U.S. Pat. No. 5,598,206 (January 1997) Bullis enabled a visual format presentation of images. An extension of these ideas was in U.S. Pat. No. 5,966,169 (October 1999) Bullis, where a larger transducer area was disclosed. However, the possible transducer area was partially utilized to enable rapid acquisition, such that focusing capability was not fully realized. The improvements also included a method of compensating pulsed signals to counter variable attenuation effects, where attenuation varied with frequency, of the human tissue medium. U.S. Pat. No. 6,368,276 (April 2002) Bullis disclosed additional improvements where very large apertures were utilized to focus very narrowly and very short depth of field was an accompanying result. Since the depth extent over which focus was effective became greatly reduced, a small focal spot was formed for any single transmit-receive event. However, as with the two preceding references, azimuth and elevation focusing was accomplished jointly through the transmit transducer array and the receive transducer array, and focusing power was not maximized. This also included a method of compensating pulsed signals to counter attenuation effects of the human tissue medium, where that attenuation varied with frequency.

A review of the past research record suggests that attenuation effects are a significant cause of focus degradation in breast tissue, especially for cases of women at the ages of greater vulnerability to breast cancer where breast tissue tends to contain a greater percentage of fat. Based on this review, methods supporting large aperture development have been invented that recognized the attenuation problem. U.S. Pat. No. 6,485,420 B1 (November 2002) Bullis disclosed attenuation leveling which enables propagation of wavefronts with uniform amplitude distribution over the wavefront. Application Ser. No. 09/975,033 Bullis, filed Oct. 10, 2001 disclosed a method of signal compensation to offset effects of frequency dependent attenuation in conjunction with an attenuation leveling configuration. While application Ser. No. 09/975,033 Bullis, filed Oct. 10, 2001 discussed a general system, that included compensation for attenuation by modification of signals, it did not include measures to accelerate image acquisition.

A further difficulty with adapting simple radar architecture to medical ultrasound applications is that air is the medium of propagation for radar signals whereas irregular human tissue is the medium of propagation of medical ultrasound signals. The radar wave signals are only slightly distorted by the air, but the medical ultrasound wave signals are significantly distorted by the tissue effects. The radar architecture, when adapted to medical ultrasound use, does not allow adequate aperture area to contend with propagation distortion effects that degrade resolution, where this causes further deterioration in disease sensing capability. There is a common expectation that larger aperture devices are more subject to medium distortion effects. This fails to correctly understand distortion effects. A new model was discussed in U.S. Pat. No. 6,524,248 B1 (February 2003) Bullis, consistent with which is expectation of a significant benefit of large aperture devices, which is to effectively capture a large area, coherent wavefront, even though it might be subject to considerable distributed interruptions.

Application Ser. No. 10/060,591, filed Jan. 30, 2002, Bullis disclosed scanning by time phasing of signals associated with elements of an array using a wavefield transformer and a bulk delay medium, thus providing for efficient phase control for large numbers of transducer elements.

### SUMMARY OF THE INVENTION

The invention is a system that was developed for medical imaging, with particular attention to breast imaging applications. It utilizes ultrasonic waves produced by a large aperture transducer configuration to emulate optical focusing with a large aperture, short focal length lens. The system architecture provides for practical operation in living tissue.

Waves are transmitted into a volume field of view to focus in spots. The volume field of view is tissue that contains a dense distribution of tissue variations, where the variations cause both responding wave signals and signal distorting effects. In this situation, responding wave signals may be from a region where sensing is intended and they may be clutter from other regions. The tissue serves as a medium that enables ultrasonic wave propagation, though the tissue causes severe attenuation of the waves.

As with high resolution focusing with a lens, very large aperture transducer arrays provide a basis for selective sensing with very high resolution. Selective sensing is in a region that is a focus spot. Focus spots are at a plurality of spot positions. For a spot position, focusing of a transducer array establishes a focal length that causes a pre-set depth to the focus spot center. Focusing of the transducer array also causes a depth of field that constrains the focus spot in depth extent. These focusing effects define a spot in the volume.

Compensation for frequency dependent attenuation by the medium enables wide bandwidth effects that function as if there was no attenuation by the medium. This compensation is arranged in relation to the focus spot. Attenuation leveling to maintain uniform amplitude wavefronts sets up a condition where all paths undergo a known attenuation process. The pre-set depth defines approximate propagation path lengths. Therefore, it is possible to predict and accurately compensate for that frequency dependent attenuation, and thus restore an intended signal spectrum. It is then possible to fully realize the benefits of wide bandwidth operation. Effectiveness of this compensation requires that the depth of field causes a spot that is small enough that compensation can be reasonably effective for a path to any point in the spot.

The selective sensing spot serves to suppress signals that arise from effects outside the spot. Such sensing is most effective when both transmit and receive operations cause focusing effects that overlay to mutually reinforce the focusing effects.

Spot focusing benefits accrue at the expense of image acquisition time, as compared with that of conventional medical ultrasound devices. A rapid scanning operation partially makes up for this penalty, where transmit-receive events are in a rapid sequence, where the transmit-receive events overlap in time. Mutual interference due to cross-over effects is suppressed by a coding method, where correlation processing is made simple by the pre-set depth that for each transmit-receive operation. A correlator channel produces a single image data sample for each spot. The compensation apparatus is combined with the coding apparatus, where coded signal waveforms are modified by compensating adjustments. Such adjustments can be applied at various points in the system, where one set of adjustments applies for a focus spot. The adequacy of a single set of adjustments also helps simplify the equipment.

Cross-over interference is further suppressed where a bistatic arrangement is used. An additional advantage of the bistatic arrangement is that switching from transmit to receive is not required and transmit events can be done at the same time that receive events occur. This enables even more overlap of transmit-receive events with reduced concern for linearity problems.

The invention includes a variety of practical device options such as with sparse array configurations hybrid beamformers, and construction details that simplify implementation. A cylindrical implementation is especially practical where large aperture benefits are achieved with an array of long, curved, elements arranged along a line. This device form then depends on mechanical motion of the transducer apparatus in a coupling fluid to sweep the lateral line throughout a three dimensional volume. Hybrid beamforming is a wavefield transformer that uses both switching and phase adjustment of signals to scan spots along a lateral line.

A key to economical and practical devices is a method of constructing large aperture transducer arrays having surface shapes that enable operation with large wavefronts that focus at spots. This method implements arrays whether elements are distributed along a line or in two dimensions.

Where arrays are two dimensional, the architectural form of this invention is amenable to combination with aberration correction methods.

### BRIEF DESCRIPTION OF THE DRAWINGS

FIG. 1 illustrates a focus spot sensing system at a point in time, where overlapping transmit-receive events are shown. Also shown is signal processing apparatus that causes focusing of transmit and receive operations, and also causes compensation for frequency dependent attenuation.

FIG. 2 describes a focal spot with lateral boundaries and depth extent determined by diffraction effects, with limits over which compensation for the spot center is effective.

FIG. 3 describes timing relationships of overlapping of transmit-receive events to produce a rapid succession of focus spot positions.

FIG. 4 describes pre-set, fixed range correlation that suppresses interference between overlapping transmit-receive events.

FIG. 5 illustrates the system in a bistatic configuration that separates outgoing and incoming waves, with a coupling fluid having attenuation matching characteristics.

FIG. 6 illustrates two cylindrical arrays in a bistatic configuration, with a barrier to provide further suppression of cross over interference.

FIG. 7 illustrates sparse array element details, where elements are arranged to cause interaction of the two arrays to provide grating lobe suppression.

FIG. 8 illustrates a hybrid beamformer that causes coarse scale movement of a focus spot by switching signals and fine scale movement of the focus spot by controlling time delays of signals, where the time delays are implemented with a wavefield transformer and a bulk propagation medium.

FIG. 9 indicates a complete bistatic system that includes separate transmit and receive apparatus, each part using a hybrid beamformer and an array.

FIG. 10 shows an adaptation of system to operate in living human tissue.

FIG. 11 shows a transducer element that is a strip formed from a card of piezo-electric material, with electrodes to enable ultrasonic operation perpendicular to the electric field.

FIG. 12 shows division of a long cylindrical strip transducer into ten individual transducer elements.

FIG. 13 shows ten individual elements formed along a straight line using a similar strip transducer method.

### DESCRIPTION OF THE PREFERRED EMBODIMENT AND VARIATIONS

The invention is designed to provide high resolution medical imaging that is incomparably superior to that achieved by conventional ultrasound technology. Furthermore, problems of conventional ultrasound technology relative to operation in real tissue must be solved.

The key objective is to resolve all possible dimensions of information to the greatest possible degree, especially where coherent conditions support such resolution. A common improvement opportunity in a variety of sensor technologies is left open by incomplete utilization of possible aperture space in conventional architecture. The medical need for much better imaging motivated exploration of such opportunities. Even though there have been significant complications, a revolutionary new architecture has been developed to enable sensing of early disease processes in soft tissue. The new architecture utilizes very large, two dimensional apertures for both transmit and receive operations. This gives the best possible resolution for viewing objects in a volume field of view.

U.S. Pat. No. 5,598,206 (January 1997) Bullis, U.S. Pat. No. 5,966,169 (October 1999) Bullis, U.S. Pat. No. 6,368,276 (April 2002) Bullis are incorporated herein by reference. These were previous concepts that utilized unprecedented aperture sizes. These inventions were structured to provide both high resolution and high speed image acquisition, though focusing power was limited by features that enhanced acquisition frame rate. The present invention is structured to use all possible focusing power of large apertures, in both transmit and receive modes, with less emphasis on acquisition frame rate. The previous work evolved signal processing methods that are used in the present invention.

While it is fundamental that very large apertures are required for high resolution imaging, and it is also fundamental that such configurations reduce depth of focus. Since a transmit-receive event only senses a small spot, time based scanning fails as an efficient system method. Thus, a high resolution system must incorporate an alternative to conventional, time based scanning. The focus spot must be moved throughout the field of view to accomplish the scan operation. Various forms of this invention use various combinations of mechanical and electronic focus spot scanning, all of which would seem to be prohibitively slow ways to operate. A necessary expediting benefit comes about from overlapping transmit-receive events. This enables much faster repetition of such events.

There is a concern about overlapping transmit-receive events that operate in a volume that contains a dense distribution of reflecting objects. In such conditions, reflections from different depths arrive simultaneously at the receiving transducer. Thus, there are many stray signals tend to obscure an intended signal. This kind of cross over interference is suppressed in the present invention by a coding system where each transmit-receive event is coded with a unique waveform. Signal processing is simple with spot focusing because there is only one range of interest relative to a given transmit-receive event and a coded signal reflection from that range will arrive at a time that is exactly known in advance. In this circumstance, replica correlation can be reduced to a simple multiplication of the received signal and a stored replica that can be carried out in real time. This real time operation can be done using simple mixer and low pass filter circuits. Two of such mixer and filter devices comprise a channel that enables acquisition of a sample of the power correlation signal that is a necessary form for image display for each spot. At least one such channel is necessary for each focal spot.

Cross-over interference can also be suppressed by use of separate transmit and receive transducer apertures that isolate outgoing and incoming paths in the medium of propagation.

As with most inventions, one of the keys to progress here is an easing of requirements. Requirements have been taken for granted for motion picture type presentations as have been accomplished by conventional ultrasound technology. While such capabilities are often useful, if they conflict with the capability to perceive early stage disease processes, then they must become secondary where that is the system objective. There is no absolute limit on frame rates by spot focused scanning devices. A variety of electronic measures speed things up by minimizing, or eliminating, mechanical scanning operations. Still, there is a fundamental handicap compared with time based scanning devices. It is expected that applications of the present invention will tend to concentrate in medical fields where movement of subjects is slow to moderate. There are electronic techniques that enable capturing of fast transient signals by relatively slow devices, especially where such transient signals are repetitive. With this in mind, it seems premature to discard the possibility of, even, cardiac imaging. However, the application to high resolution breast imaging is very much an achievable objective, and this is the primary context of the remaining discussion.

This new architecture is particularly meaningful for a focused apparatus where f is less than 4 and focal length to depth of field ratio is more than 10, where depth of field is the range extent where diffraction effects control focusing resolution. Preferred applications involve focusing where f is closer to 1 or less and that ratio is more than 100. As f goes from 4 to 1, scanning in range based on time of arrival goes from an awkward method of image acquisition to practically impossible, so this becomes the regime of the present invention.

Of particular interest to the breast imaging application, the new device also provides capabilities that make operation effective under difficult conditions caused by propagation characteristics of breast tissue. Attenuation leveling is included such that focusing is not degraded by variations in amplitude of signals over large focused wavefronts.

While attenuation leveling serves to maintain large, focused wavefronts as they propagate, the waves still undergo significant attenuation, where an attenuation rate varies with frequency. This attenuation rate determines attenuation at a frequency that is proportional to path length in the applicable medium. The detrimental effect of such attenuation is to degrade bandwidth benefits that enable time resolution and to cause in a shift of the operative band center that degrades the diffraction limited focusing effects. Compensation is applied to signals to reverse these effects. Signals are still attenuated, but they are evenly attenuated so that uniform bandwidth effects are enabled. Such compensation can be applied to signals prior to transmission or after reception. In either case, the necessary adjustments depend on both the frequency component to be adjusted and the details of the propagation path to be encountered. Leveling enables knowledge of such path details. The focus spot size is designed to enable compensation adjustments that are the same throughout the spot. This is important whether compensation is applied to signals before transmission or after reception. Compensation must be applied where system operations are linear and adjustments can be applied to individual frequency components of signals.

Other features of the invention include sparse transducer configurations that enable economical fabrication of systems, especially for systems in a form suitable for breast imaging.

The architecture is structured to be compatible with methods that cause aberration correction.

The remaining discussion addresses details with reference to the drawings. A general comment is that signal paths shown on drawings are generally shown as simple lines. Such lines should be understood to include amplifiers, filters, impedance matching transformers, and any other such devices necessary to properly implement the ideas indicated, as would be expected of persons skilled in the respective arts involved. The discussion is carried out at the system design level so as to reasonably convey the concepts in the invention, yet sufficient detail is provided to enable implementation by an appropriate organization. Previously cited patents are useful references. These references therein are incorporated in this present specification by reference. The present document takes precedence in case of conflict.

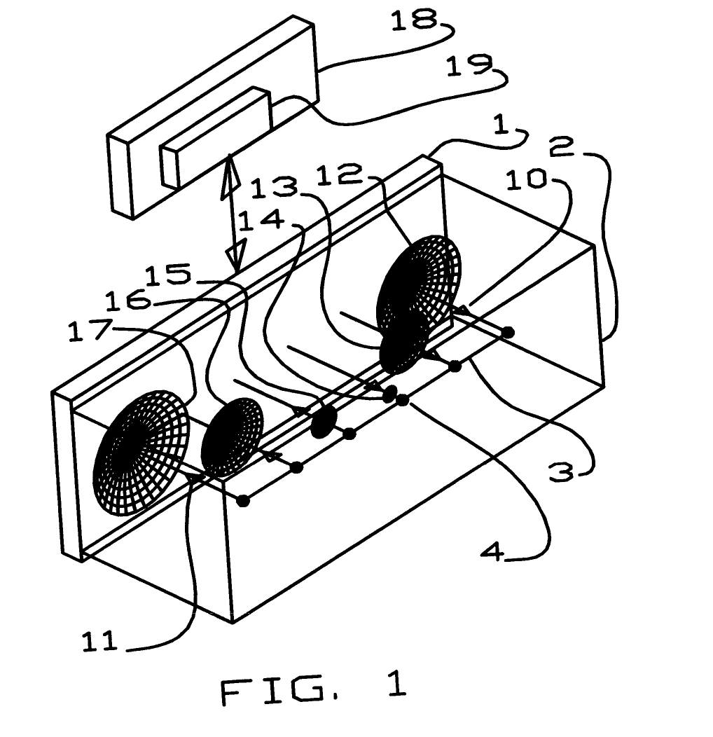

FIG. 1 illustrates formation of six focal spots arranged along a line 3 by a general transducer apparatus 1 that transmits and receives. Spherically shaped wavefronts are represented by mesh surfaces that indicate spherical surfaces. The circular form of the mesh is only an approximate representation of waves that would actually be produced by apertures that are not, in general, circular. This illustration captures conditions at a single point in time that is just after transmission of a wave indicated by a wavefront 12 in a volumetric medium 2 that is formed by the transducer apparatus in conjunction with signal control of signals that drive the transmitting operation. Wavefronts are shown as mesh surfaces, only to convey the concept. That transmitted wave travels along an outgoing path 10. An immediately previous transmission caused a wave indicated by a concentrating wavefront 13 and before that a much concentrated wavefront 14 that is about to arrive at a focal spot 4. These represent three separate transmit events which enable reflections and then respective separate receive events. Since reflections only from within the spot are of interest, the receive event is brief. Each transmit event is paired with a receive event to be a transmit-receive event. The concentrated reflected wave 15 results from a yet previous transmission and an enlarged reflected wave 16 is associated with an even earlier transmission. The largest reflected wave 17 is about to be received by the transducer apparatus after having traveled along an incoming path 11. This will complete the transmit-receive event that was the first, among those shown, to start. The reflected waves are shown in a simplified form that does not fully represent the spreading of reflected waves and focusing of received waves serves to selectively receive signals from the focus spot. The illustration shows the overlapping which is necessary to accelerate the focal spot scanning operation. The illustrated single point in time showed six simultaneous events in their particular status at that time. The arrangement along the line 3 is only representative of more complicated arrangements that would cause spots that covered that line 3. The actual number of simultaneous events would be maximized to give the fastest possible scan.

FIG. 1 also shows signal processing apparatus 18 that operates to control signal transmission and to receive signals. The signal processing apparatus 18 includes compensation apparatus 19 that operates to compensate for frequency dependent attenuation over paths to the spots.

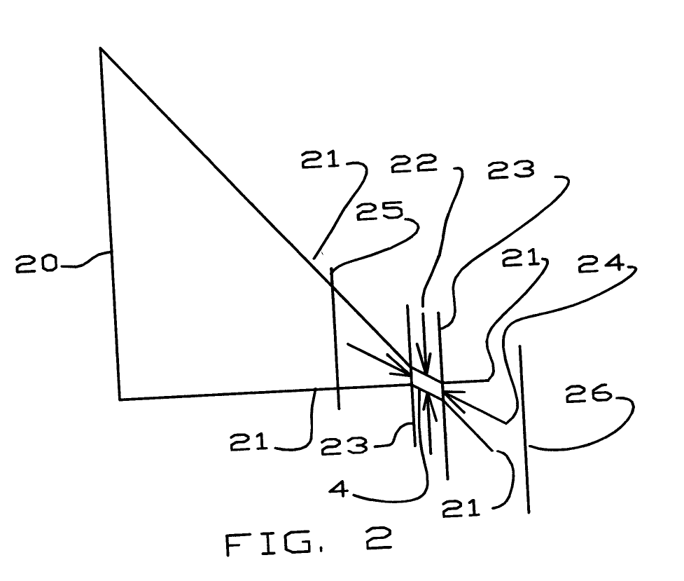

FIG. 2 defines the focal spot to be the diffraction limited region in approximate system terminology. A vertical aperture extent 20 causes geometric focusing bounded by intersecting lines 21. Diffraction prevents the focal spot from being any smaller than the separation indicated 22. Outside the diffraction limited zone, geometric focusing rules apply. The transition is indicated by vertical lines 23 that define the depth of field that is the separation 24 between these vertical lines 23. It can be seen from this how a short vertical aperture extent compared to that shown 20 and widening of the diffraction limited vertical dimension compared to that shown 22 would cause elongation of the depth of field. A very large depth of field develops rapidly since the vertical dimension of the diffraction limited zone is inversely dependent on the vertical aperture extent. This enables operation over an extended depth with a simple system and this fits well with the use of time based scanning in depth. While this equates to a strong practical concept, the characteristics of ultrasonic waves together with this simple system means that medical ultrasound of this type will not achieve the high resolution that is needed. This is a barrier that must be broken by a new architecture that abandons these practical advantages, but very important benefits are also realized. The first is that the diffraction limited zone can be made as small as needed and the second is that depth of field focusing effects can be used to advantage. A critical advantage is to aid in the prevention of interference by the overlapping transmit-receive events.

FIG. 2 also shows the limits over which compensation for attenuation is effective, where such compensation is based on a calculation for attenuation effects to the spot center. The operating regime of the invention is for focal lengths, and the spots they define, that stay within these limits. Compensation is, in general, computed for each spot. Compensation involves modifying signal frequency components, where the modifications are inverse to attenuation over paths to the spot for both outgoing and incoming waves. In many situations this is enabled by attenuation leveling, where the attenuation leveling apparatus is required so that the material medium of the propagation paths is known. This enables calculation of the compensating adjustments. Since the adjustments depend on depth, they will gradually lose effectiveness for points that are further away from the spot center. The limits 25, 26 indicate the extent of the effective region for the illustrated spot 4.

Overlapping transmit-receive events cause significant problems in a medium that contains many distributed tissue variations that cause reflections. While reflections are arriving from the range of a spot, many other reflections are arriving from points at shorter and longer ranges. Such interference signals would be a significant cause of degradation of image quality, except for the measures that are used to suppress interference between the events. The depth of field effect helps suppress arrival signals that come from ranges that differ from the range of the intended spot. Another method is a signal code filter that utilizes distinctive codes for successive transmissions, with single sample replica correlation processing that extracts one power sample from a collection of interfering received signals, for a pre-set range. The single power sample senses one code while suppressing response to other codes. This pre-set range form of correlation is a dramatic simplification of known replica correlation apparatus, since it operates in real time using only a multiplication-integration process.

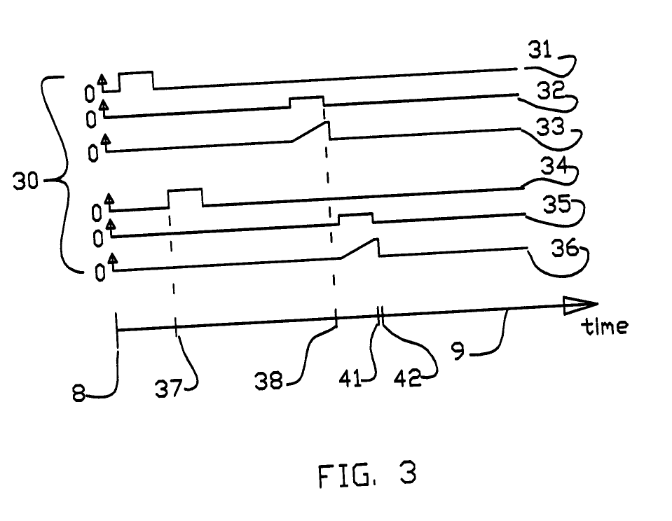

FIG. 3 illustrates two overlapping transmit-receive events relative to a time line 9 having an arbitrary starting point 8. Activity is indicated by traces 30 that rise in a positive direction as indicated by the arrows. The earlier of two transmissions is indicated by the first pulse of the top trace 31. It has a duration indicated by the length of that pulse. It has a corresponding receive event shown below 32. The ramping of the trace below 33 indicates growth of the integrated result of a multiplication between the received signal and the replica of the code, where that multiplication begins at the pre-set time at which the arrival from the focal spot is expected. Overlap means that the transmission time 37 of the second transmission indicated by the next trace 34 begins before completion time 38 of the first reception. Between a point in time 41 when the integration output 36 for the second receive event stops increasing and a point in time 42 when a reset is done, the system samples the power level which is directly related to the desired image signal for that spot.

Though not strictly required, a practical provision is to implement the system such that transmit events do not overlap receive events so as to avoid electrical crossover coupling. It is possible to greatly accelerate operation if transmission events themselves overlap. Coded signals are useful in regard to these arrangements. Uses of simultaneous beams that are respectively coded was discussed in U.S. Pat. No. 5,598,206 (January 1997) Bullis.

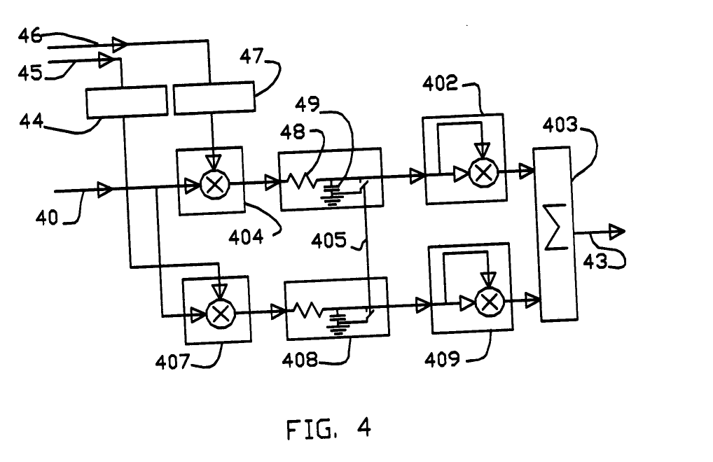

FIG. 4 shows system level details of the configuration of a channel to carry out pre-set range correlation. Two replica signals are held in storage 44, 47, having been transferred 45, 46 from the same base that is used for driving the transmit signals. These two replicas are the same waveforms except for a very small time delay that is necessary to take a power sample. Both are delayed relative to the transmission of the same waveform by a time that equals propagation time of the wave to and from the focus spot center. Exact timing must account for electronic delays. The signal from the transducer system is conveyed 40 to parallel multipliers 404,407. These multipliers are familiar mixers, where the designer may choose between balanced mixers built with diodes or Gilbert cell type mixers. Because the integration is long term and the desired output is almost a dc value, the mixer can be allowed to put out harmonics with little concern. An integration process in the two halves of the channel is shown in one place 408. The integration operation in one half of the channel is indicated by a resistor 48 and a capacitor 49. The other half operates the same way. The reset switches are operated 405 at about the same time. The needed power sample 43 is the sum 43 of the squares computed by multiplication of a signal times itself 402, 409. This pre-set range correlation process provides one sample for each pre-set range. It is simple to implement since the incoming signal is processed in synchronism with its actual reception. If more than one power sample is appropriate to sub-divide spots, parallel channels can be used, with slightly delayed replica signals to cause slightly delayed samples.

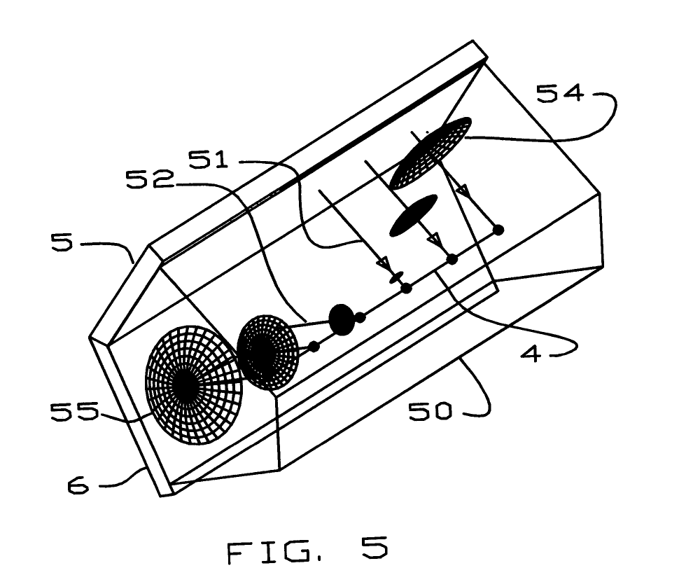

A further measure that suppresses interference between overlapping transmit-receive events depends on angular based exclusion effects of focused beams that are separated by a bistatic arrangement. In this arrangement there is a bistatic angle between an incident wave at an object and a reflected wave at that object. The bistatic angle has a subtle effect on image results that varies as the angle varies. This arrangement has a further advantage in being capable of operating without switching transducers from transmit to receive mode. FIG. 5 shows spots arranged along the line 4 similarly to the arrangement of FIG. 1 except here the transducer apparatus is specifically separated into a transmitting part 5 and a receiving part 6. As before, the illustration represents status at a single point in time that is shortly after the most recently occurring transmission that caused the wavefront 54. The wavefront 55 that is about to be received was the first of the six transmit-receive events involved. An outgoing path 51 and an incoming path from that same spot, where this incoming path is parallel to the incoming path 52, define the bistatic angle for that spot. For a transmit-receive event, the transmit wave and the part of the reflected wave that becomes the receive wave operate in two separate spaces except when the two spaces are very close to the focus spot. This minimizes spatial overlap of transmit and receive waves and prevents cross-over of early and late signals from transmit-receive events that overlap in time.

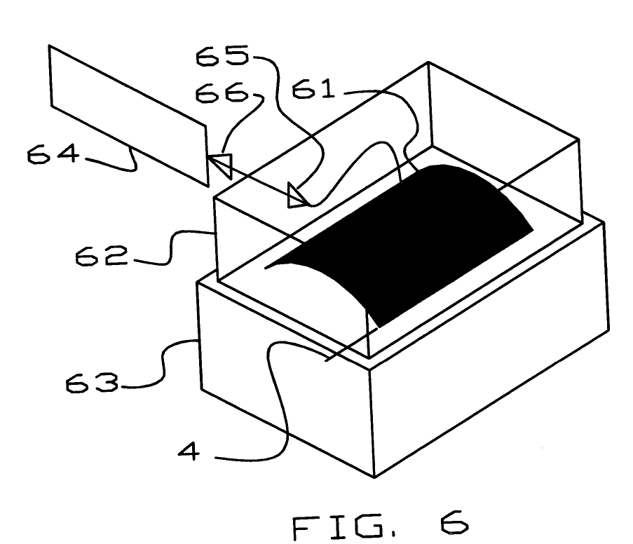

FIG. 6 shows an efficient form of large aperture transducer 61, The illustrated device is used for both transmitting and receiving. The cylindrical array has an axis that is the line along which focal spots are arranged, where that line is interior to the patient as represented by the lower box 63. This array is formed with strip transducers that are shaped to fit the cylindrical surface contour of a backing block. The radiating cylindrical surface 61 is all that is shown in the figure, where the many, very thin strips show as a solid black surface. The electronic system 64 provides outgoing 65 signals for transmission and processes incoming 66 signals. Each element is focused due to its cylindrical shape in one dimension. Focus in the orthogonal dimension, either for transmit or receive, depends on the electronic system, where delays must be appropriately applied to signals to and from the transducer to achieve the desired focusing effects. Electronic beamforming is well known as evidenced by the early patent, U.S. Pat. No. 3,039,094 (June 1962) Anderson. It is incorporated herein by reference. A later illustration in the present disclosure makes it graphically apparent how such beamforming devices must function.

In FIG. 6, the upper box 62 is a container filled sufficiently to immerse the radiating surface of the transducer 61 with coupling fluid. The coupling fluid is designed to match body tissue in respect to ultrasonic propagation characteristics. It is specifically required to match the frequency dependent attenuation characteristics of the tissue 63. Eagle brand evaporated milk by Borden was found to be a good match to breast tissue, as specified in inventions U.S. Pat. No. 5,625,137 (April 1997) Madsen et al. and U.S. Pat. No. 5,902,748 (May 1999) Madsen et al. where it was used to form simulated human body parts called phantoms. Instructions for use of the evaporated milk and other materials and formulations can be found in all these inventions that are incorporated here-in by reference. The same material was applied in the invention U.S. Pat. No. 6,485,420 (November 2002) Bullis for the purpose of coupling ultrasonic waves between a transducer device and a body part. This latter invention describes arrangements of devices to enable the coupling function. There are other material possibilities that may be substituted, though some cause undesirable reflections. The general requirement is to match attenuation such that waves travel in a way that an intended amplitude distribution over the wave is not disturbed as the wave propagates, except for its general amplitude. This is not meant to preclude use of attenuation effects in the medium to cause desirable taper of the amplitude distribution, where this can beneficially affect sidelobe performance. These prior inventions are incorporated by reference.

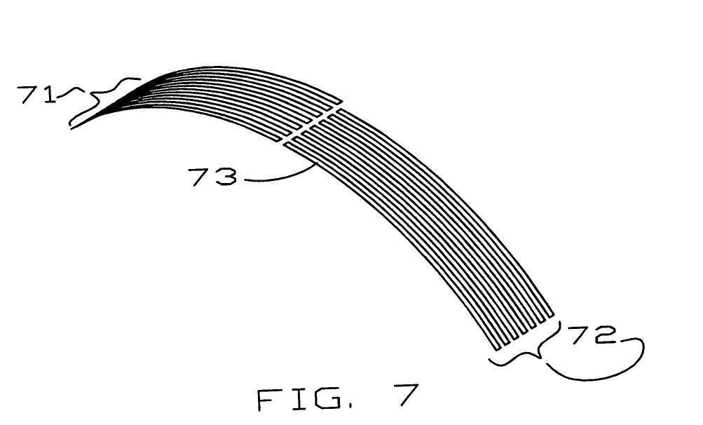

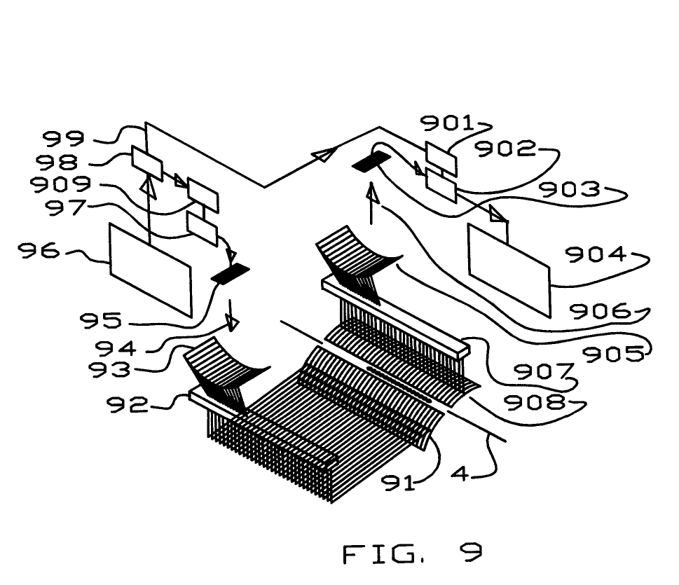

Radiating surfaces of strips are shown in FIG. 7. This shows the way the strips are arranged for the cylindrical form of FIG. 6 except FIG. 7 shows separation of the transducer array into transmitting 71 and receiving 72 parts. This implements the bistatic form as discussed relative to FIG. 5, and to be later discussed relative to FIG. 9, where transmit and receive functions are separate. An example active surface 73 is outlined. For both parts, a gap is left between adjacent strips and the spacing is greater than half a wavelength such that there is a grating lobe issue, where grating lobes are spurious responses of an array, whether for transmit or receive. Since there is a significant advantage in allowing such sparse distributions, there is strong motivation to fix the grating lobe problem. Close examination of FIG. 7 will show that the spacing between strips is different for the transmit side 71 than it is for the receive side 72. This causes transmit grating lobes and receive grating lobes to be at different angles so reflections caused by transmit lobes will not be sensed by receive lobes. The continuous strip will not cause grating lobes in the orthogonal plane. It will also fix the focus point on the cylinder axis.

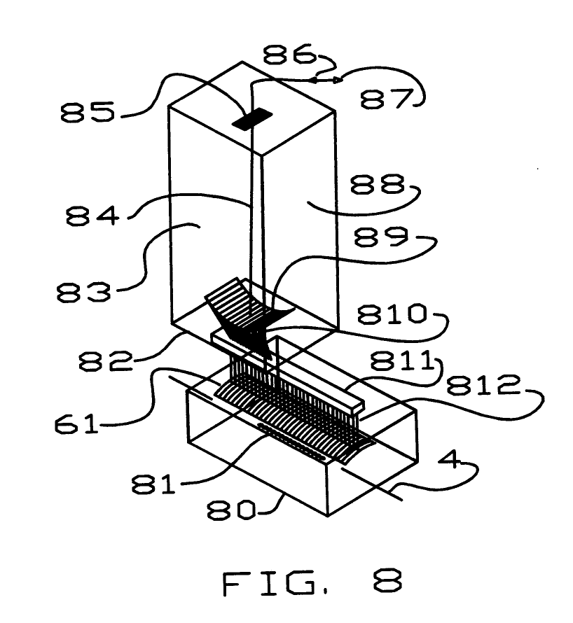

FIG. 8 implements the general form of FIG. 6, where the array 61 is the same and the line 4 is the same. The box 80 in FIG. 8 indicates the combined propagation medium made of the patients body 63 and the coupling fluid container 62 of FIG. 6. The apparatus shown with the array is a wavefield transformer implementation of the functions indicated by the block 64 of FIG. 6 according to the pending invention application Ser. No. 10/060,591, filed Jan. 30, 2002. In the illustrated example of FIG. 8, the beamforming is done both for transmit and receive by the same device. This is a hybrid device that combines a stepping operation, like the operation known as “linear array” operation in the ultrasound community, and an angular beam steering operation, like the operation called “phased array” operation by the same group.

Simplified operation to cause spot focused outgoing waves begins with conveyance 86 of a coded signal waveform to an element of a small, flat array 85 of transducers. That element causes an ultrasonic wave to radiate in a fluid medium 83 in a container 82 along a line 84 toward a shaped array 89, where the shaping is not, as it might appear, to focus on the source element, but rather, to provide signals that go on lines 810 with delays that cause waves transmitted by the larger array 61 to be shaped to focus at a spot 81 along the line 4 shown. For a first coarse increment spot position 81 along the line 4 the lines 810 are connected through lines 812 to respective elements of the large array 61 beginning with the first element through a switching device 811. A second coarse increment spot position is arranged by switching with the switch device 811 that reconnects lines 810 to respective elements of the large array 61 beginning with the second element. The spot is thus moved in coarse increments along the cylindrical array axis 4. The switching device is implemented with high speed CMOS devices such as the Motorola MC74HC4051 along with appropriate timing control and amplifier circuitry. These are especially appropriate device since they switch signals with average levels of zero. For each coarse spot position, multiple fine increment spot positions are necessary. This is related to the sparse spacing of elements in the large array 61. Spots are moved to successive fine increment positions by conveying 86 coded signals to successive elements of the flat array 85. This fine increment method functions like a simple optical lens, where the small flat array corresponds to the object plane in the optical system and positions along the line 4 correspond to the image plane in the optical system.

The above described process is reversed to caused spot focused operation that selectively senses incoming, received waves. This ultimately results in conveyance 87 of waveforms to the pre-set range correlator.

Container walls 88 are lined with absorber material that is shaped to prevent effects of spurious responses or sidelobes of either array in both directions.

Although all possibilities for parallel operation of channels are not detailed here, it is deliberately suggested that such possibilities are intended. Parallel operation as with imaging in optics is possible. As taught in U.S. Pat. No. 3,039,094 (June 1962) Anderson, receive beams can be easily operated on a simultaneous basis. The fluid based time delay adjustment device in the container 82 would be operated in the linear range so that waves would propagate superimposed on each other. Multiple copies of the switching device 811 can be attached to the array 63 in parallel. With transmit beam coding, as taught in U.S. Pat. No. 5,598,206 (January 1997) Bullis, U.S. Pat. No. 5,966,169 (October 1999) Bullis, and U.S. Pat. No. 6,368,276 (April 2002) Bullis, parallel transmit operations are also possible. Although these methods can greatly improve acquisition frame rates, the first preferred embodiment of the present invention does not implement such parallel operations. Rather, highest possible quality imaging using sequential focus spots is the present primary objective.

Ideally, the design would use all possible focusing power of the transducer aperture in both directions. This is most completely done where all the available physical space is occupied by one very large aperture that is used for both transmit and receive operations, as shown in FIG. 8. Where elements are spaced to be half a wavelength on center, the switching system that steps the beam will directly accomplish the fine scale movement of the focus spot. The time delay apparatus 82, or its electronic equivalent, will still be needed, except the small upper array 85 will be reduced to a single element. Here the switching device 811 gets large in order to provide a large field of view. These considerations lead to trade-offs that result in the configuration of FIG. 9, which is better suited for immediate implementation.

Other embodiments include extension of the hybrid beamformer for arrays that are arranged along a line to a hybrid beamformer for arrays that are arranged along a plane. In this variation, the switching device 811 is expanded to connect to the many times more elements that are produced when the strip elements of the array 61 are subdivided. Correspondingly, the number of time adjusted signals is much greater, so the number of elements in the array 89 is increased by sub-dividing the elements shown.

FIG. 9 shows a double array 91,908 configuration, which gives grating lobe suppression so that it is not detrimental to use sparse arrays. The method of grating lobe suppression was explained in reference to FIG. 7. Focus spots are larger since the apertures are individually smaller than the single large aperture of the two way transducer system. It is not completely clear which will ultimately prove to be the best system.

The arrangement of FIG. 9 suppresses cross-over interference by isolating transmitted waves from that section of responding waves that the receiving aperture will capture. A barrier panel may provide additional suppression, where the barrier panel is in the propagation medium between the transmit waves and the, to be captured, responding waves. This would be an absorbing material, with surface shaping to enhance absorbing effects. Design of the panel is such that it would minimize diffraction.

System details are shown in more detail in FIG. 9. Many are applicable to all system forms discussed. Generation of coded waveforms 96 includes production of unique codes. Golay codes are known to be desirable in this context, as indicated in U.S. Pat. No. 4,855,961 (August 1989) Jaffe et al., which is incorporated herein by reference. However, a very simple way to generate codes is to operate a random number generator to obtain a multiple signal sample sets that are a series of numbers, where statistics for generation of all sets is specified to have zero mean and fixed variance. Each resulting number set is a unique code. Full cross correlation functions between the various sets are used to reject unacceptable codes, where any peak that is an exceptionally high correlation amplitude would be cause for rejection of a code. This can all be done in advance so that computation time is not critical to real time operation. The surviving number sets are then both unique and mutually non-correlated to a reasonable degree. Codes are stored 98, replicas of codes are communicated 99, and stored 901 for appropriately timed readout into the pre-set range correlator 902.

Codes are modified 909 to compensate for frequency dependent attenuation, according to the invention of application Ser. No. 09/975,033 Bullis, filed Oct. 10, 2001, and stored for appropriately timed readout as transmit signals. These codes are specifically formulated for the spot system configuration.

To implement attenuation compensation, signals are adjusted in amplitude such that propagation attenuation over the path to the spot is reversed to achieve the intended shape of the broad band signal spectrum after reception. The frequency components in the spectrum are modified to have the opposite effect as the attenuation, and this is enabled by prediction of the attenuation effect based on knowledge of the medium effects to be encountered over a path to the focus spot center. Phase adjustments are also included to compensate for phase shifts due to velocity dispersion over the frequency band so as to make the frequency response linear. This form of compensation works well with broadband codes that have long duration. Gaussian shaped pulses are used in conventional systems to minimize degradation by attenuation, but this method is not useful with coded waveforms. The compensation approach can also be implemented using filters having appropriately shaped frequency response functions. Compensation can be also be applied after reception of signals, though signal to noise ratio effect should be considered for this mode. The preferred method is to use an arbitrary waveform generator such as the HP 8770A, Agilent, to store and play back a signal for transmission that is set up in advance to produce the desired waveform. This instrument is used in connection with GPIB computer interface card by National Instruments along with software interface by National Instruments and Basic programming language written by the user according to the HP 8770A documentation and the National Instruments GPIB documentation. These are representative devices that enable low cost construction of early test models, but they have counterparts that are directly integrated into present day computers of the PC type. A basic system will include one such arbitrary waveform generator that produces compensated and coded signals for transmission, and two such generators that produces uncompensated, coded signal replicas that are multiplied with received signals beginning at a time for arrivals from the spot center. Multiplied results are integrated over time. There are thus two integrated outputs that are to be individually squared and summed together, as previously discussed in respect to FIG. 4.

Alternatively, compensation can be implement at various points after reception. It is convenient to do this after summation of received signals and before conversion to a power form that becomes an image data signal for display. A filter having frequency response that reverses the frequency dependent attenuation effect for the associated spot will be appropriate. It must have a linear phase response over that band to maintain phase relationships of the intended broad band signals.

Devices 93, 94 that provide time delayed signals resulting from wave propagation 94 operate as described in reference to FIG. 8, except here it is only one way as indicated by the propagation direction 94 arrow. Similarly, devices 905,903 operate in the opposite direction as indicated by the propagation direction 906. Containers and fluid associated with these devices that apply appropriate signal time delays are not shown, but are the same as the container 82 and fluid 83 shown in FIG. 8. The switch device for outgoing signals 92 is one way and carries only transmit signals. Similarly, the switch device for incoming signals 907 is one way and carries only receive signals. After correlation processing 903 signals are presented as images 904.

Another future variation is an extension of the bistatic architecture to arrays where transducers are arrayed in two dimensions, that is similar to the extension of the two way architecture that would utilize one such two dimensional array. Both of the arrays would, in this case, be flat.

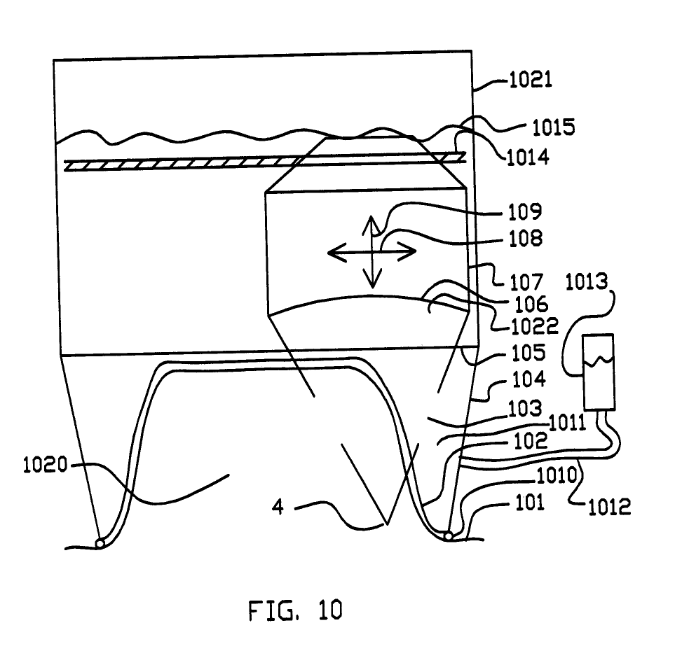

FIG. 10 shows operation of a transducer device with details of the adaptation to medical examination purposes. Here the radiating surface 106 corresponds to the radiating surface 61 of FIG. 6 or FIG. 8. In the view of FIG. 10, the line along which spots are focused 4 shows as a point 4. The representative tissue 63 of FIG. 6 is specifically a female breast 1020 that has a tightly conforming barrier surface 102 of latex or such, with seal 1010. Seal 1010 is a hollow tube that rings the body part and is connected to means to cause vacuum that causes the conforming effect such that the barrier surface 102 isolates the skin 101 from the attenuation leveling material 1011. Containment of the leveling material is due to container walls 104. When this device is pressed against the chest over the breast, the vacuum draws the barrier, with fluid next to it, against the breast, whatever the actual breast size and shape might be. Fluid reservoir 1013 provides necessary fluid to flow. An idealized wavefront is bounded by an interrupted line 103 and its opposite. A fairing surface is a mylar sheet that is stretched to be an approximately planar surface that flattens the body part sufficiently to facilitate mechanical scanning, and establishes an upper fluid compartment that is separate from the lower compartment. Container walls 1021 hold the coupling fluid in the upper compartment. The upper surface 1015 of the upper coupling fluid is a free surface. Ideally the fluids are the same in the two compartments. It is allowable to use upper compartment fluid 1022 that does not match attenuation, for reasons of convenience, but power levels must be adjusted as the vertical scanning 109 establishes shallower depths in the body part. Without such adjustment, the power intensity at the focus point could easily become excessive and dangerous. Electronic scanning causes the focus spot to move along the lateral line 4 into the plane of the drawing. Mechanical scanning moves the transducer apparatus in the orthogonal lateral direction 108, where the illustration shows this scanning direction to be implemented by a lead screw arrangement 1014. Vertical scanning 109 requires another lead screw arrangement that is not shown.

To aid in application of the device, an auxiliary sensor is included to sense peaks in blood pressure with capability to use this sensed information to arrange scanning that is synchronized, or shifting in synchronism, with motion of tissue.

A critical part of spot focusing systems is the transducer apparatus. Large area surfaces having prescribed shapes are needed. Such are not readily available. An economical concept has been developed that includes a manufacturing process that can be carried out with relatively inexpensive tools.

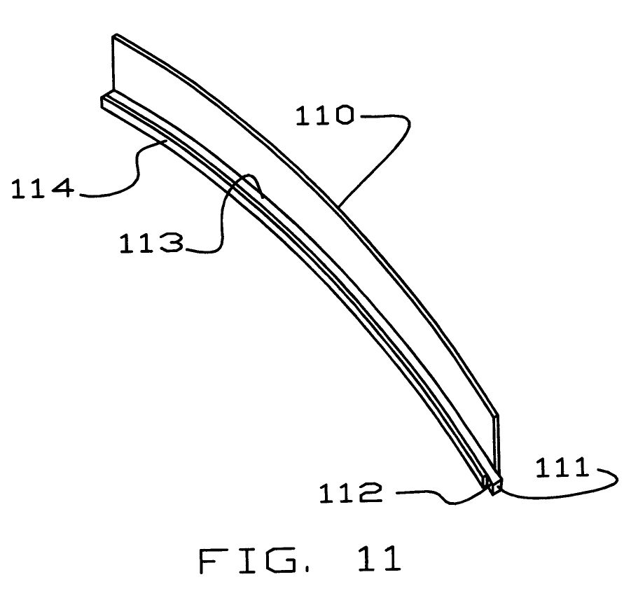

The large aperture transducer is constructed of elements that are in an arrangement that is fixed by attachment to a backing block surface, where the backing block surface provides both rigid support and an absorbing function. A special feature of the present invention is a strip transducer concept. FIG. 11 shows construction of a strip transducer element that is required to implement a radiating surface 73 as shown in FIG. 7, where that radiating surface 73 is opposite the surface 113 shown in FIG. 11. Bonding of the surface 113 to the shaped backing block surface is done with epoxy. The backing block is also epoxy, having been previously cast as a block and machined to give an accurate surface. A high performance epoxy is supplied by Abotron, Inc., where the specific formulation is in proportions of 10 parts Abocast 50-24 to 11.5 parts Abocure 8501-4 by weight. This is a good material to work with since pot life is many hours at room temperature, and curing is accomplished at an acceptable, 100 degrees centigrade. The active transducer material is piezo-electric, which is not significantly deteriorated in capability by exposure to this temperature. The piezo-electric ceramic material is cut from cards that are 2 inches by 3 inches by 0.0075 inches. These cards are called plates and have metalized surfaces on both of the 2 by 3 inch flat sides. Suppliers of such parts include Morgan Electroceramics. The metalized surfaces of the supplied parts must be built up by electroplating of 0.0004 inches of copper. A system based on an additive by Lea Ronal resulted in an even surface. The plating operation is modeled after printed circuit board methodology. Parts indicated 110,114 are made with the same Abotron epoxy used for the backing block, except micro-balloons are added. The microballoons are Scotchlite glass bubbles, H50/10,000 EPX supplied by 3M Corporation. The formulation used is 10 grams Abocast 50-24 to 11.5 grams Abocure 8501-24 to 11.5 grams glass bubbles. This gives a very viscous mixture that rolls out between Teflon sheets to reasonably accurate thickness using rails and a rolling cylinder. With curing at 100 degrees centigrade, thin sheets of material are thus produced. These are surface ground using a milling machine with a diamond end mill to give a precise thickness. These plates are then bonded to the piezo-electric plates to make a 2 by 3 inch sandwich. Then the strips are cut in a computer controlled milling machine with a diamond end mill to yield the very thin strip of piezo-electric ceramic material 111 and the very thin strip of glass bubbles and epoxy 114 while leaving a wider strip 110 of glass bubbles and epoxy on the opposite side. The wider strip fits into a groove that was milled into the backing block. Using the same epoxy mixture, without the glass bubbles, this strip assembly is then bonded in place, such that epoxy forms continuous connection between the piezo-electric strip and the epoxy backing block. The piezo-electric strip 111 protrudes sufficiently to leave a tab that exposes the copper surface 112 to enable soldering of wires to this side and the opposite side of the strip. This creates electrodes that enable imposition of an electric field that is controlled by electronic circuitry to connect to signals. Many such strips are cut from a sandwich, depending on the exact dimensions of the strip and the diamond end mill. The operating frequency depends on the width and thickness of the piezo-electric strip. Achieving the desired bandwidth and center operating frequency is a capability of the ultrasound industry, though guidance is available from the supplier of the piezo-electric cards. For long strips 111, the length is not a significant factor in determining operating frequency. Unlike the usual practice in medical ultrasound transducers, operation of this transducer causes ultrasonic displacement that is perpendicular to the direction of the electric field. The curved form of this element enables formation of an array of many such elements to enable focusing of spots along a line 4 such as that line shown in FIG. 9.

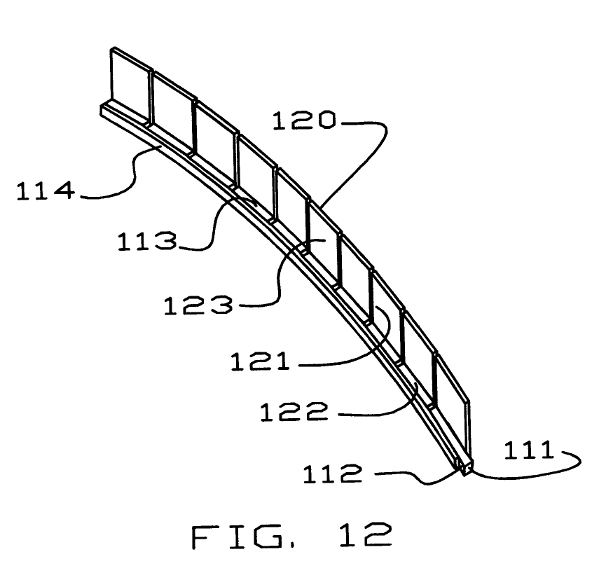

To enable some electronic control over position of that line 4, it is necessary to divide the strip into smaller elements. The assembly will then be a two dimensional array of transducer elements. FIG. 12 shows such a divide strip. It has the same glass bubble and epoxy strip 114 and the same piezo-electric strip 113. The same tab protrudes to expose a metal electrode surface 112. However, cuts like the cut 122 are made in the wider glass bubble and epoxy strip 120 that also go through most of the piezo-electric strip, while preserving continuity of the copper that is an electrode for all the elements 122 that are formed. Since the cut went through the back side copper layer, the elements have separated electrodes on that back side. These are left as exposed metal like the exposed metal on the tab 123. The cuts are made with a diamond end mill cutter or a slitting saw. Precise machining is required to leave sufficient copper on the tabs 123. The strip assembly will depend on the front glass bubble and epoxy strip to control position of the elements when the strip assembly is attached to the backing block. Wires are attached to the copper tabs 123 as well as the exposed surface 112. The wires attached to the individual tabs must pass through holes that are drilled in the backing block. The wires are like those in the next figure.

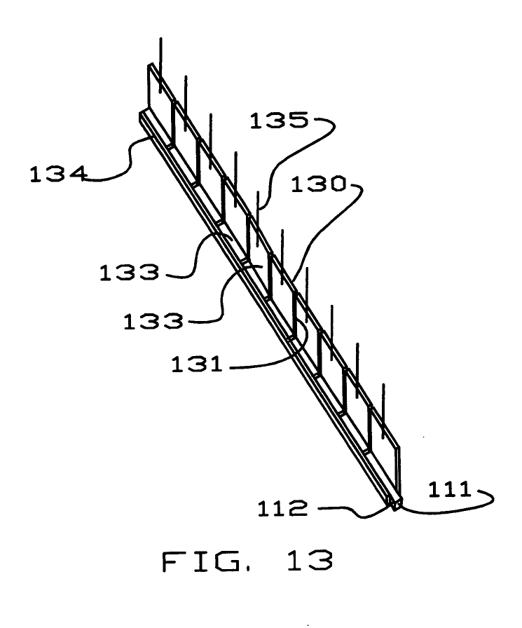

Rather than a subdivided curved strip, a subdivided straight line strip is indicated in FIG. 13. This shows the previously described wires, like the wire 135 that is attached to an individual element electrode 133. A wire is again attached to the same common protruding electrode surface 112. An individual element 133, of the ten formed along the strip, is also shown. This sub-divided strip is constructed like the curved strip assembly of FIG. 12. This is the form necessary to implement the more general devices of FIG. 1 and FIG. 5 where flat transducer apparatus are shown. These flat arrangements require signals of adjusted time delay to form the focusing effect in two dimensions. This is the purpose of the two dimensional version of the wavefield transformer that was illustrated in one dimensional form in FIG. 8.

By the examples of this straight line strip assembly and the curved strip assembly the general applications of the technique are made apparent, where a variety of transducer shapes and a variety of element distributions can be implemented Not only do the two dimensional arrays enable electronic control of focus spots such that mechanical positioning is not necessary, they also enable further features that include aberration correction methods. One such aberration correction method was disclosed in U.S. Pat. No. 6,524,248 (February 2003) Bullis.

1. An imaging system that is adapted to image objects in a selected object region in a volume, where said volume includes a medium of propagation that enables ultrasonic wave propagation in said object region, where objects in said selected object region cause ultrasonic effects, and where said medium causes frequency dependent attenuation, where attenuation varies over operating frequencies, where said imaging system includes transducer apparatus that transmits and receives waves that propagate in said medium, where said transducer apparatus enables spot focusing of propagating waves at focus points in said selected object region, where said spot focusing enables approximately exclusive sensing in spots, where a spot is a volume region about a focus point that is enclosed by diffraction limited effects that result from focusing of a propagating wave, and
apparatus that operates to cause transmit-receive events for respective said selected spots, where a transmit-receive event includes transmitting of signals to form a focused outgoing wave that is focused for said spot and receiving of signals to cause focusing, for said spot, of incoming waves, where operation of said transmit-receive event enables acquisition of signals that arise from ultrasonic effects that occur in said spot, and apparatus that operates to control positions of said focus points to cause a collection of said spots that approximately continuously covers said selected object region in said volume, where overlap of said transmit-receive events enables rapid scanning of said collection of said spots, and
apparatus that compensates for said frequency dependent attenuation, where compensation causes compensated signals that are associated with said spot, where frequency components of a compensated signal are constant in amplitude over said system operating frequencies, where said compensation is determined for a point in said spot and where size of said spot is sufficiently small that said compensation is effective for any point in said spot, and
apparatus to form image data using such compensated signals from said collection of said spots.

2. An imaging system according to claim 1, where said approximately exclusive sensing serves to suppress signals that arise from points outside a said spot.

3. An imaging system according to claim 1, and apparatus that enables broad band operation for purposes of resolving signals in time.

4. An imaging system according to claim 1, where said compensation applies corrections to signals prior to said transmission of signals, such that transmitted signals are pre-compensated signals that enable said compensated signals.

5. An imaging system according to claim 1, including apparatus that applies said compensation after reception to produce said compensated signals.

6. An imaging system according to claim 1, including apparatus that operates to compensate signals for apparatus transfer functions that cause output signals that have a distorted frequency spectrum, where distortion corrections enable output signals with a relative frequency spectrum that is approximately the same as a relative spectrum of a corresponding input signal.

7. An imaging system according to claim 1, including code apparatus that enables coded signal operations that enhance said approximately exclusive sensing, where unique codes are generated for respective spots, where for said spot, a unique code is used to generate a coded signal and a coded replica signal, and said coded signal is carried through said system such that said system acquires coded, compensated, signals from said spot, and where said coded replica signal is held in storage, and said code apparatus also enables pre-set correlation of waveforms, where a waveform is a signal amplitude as a function of time and pre-set correlation includes multiplication of respective amplitudes of two waveforms, that are in a pre-set time relationship, to obtain a product waveform and integrating said product waveform, and pre-set correlation is operated to compare said coded replica signal with said coded, compensated, signals from said spot, where said multiplication begins at a fixed time that is a time of arrival expected for a compensated signal from said spot, where said correlation produces an integration output that is a correlated signal sample from said spot, and said correlated signal sample is utilized in producing an image data sample.

8. An imaging system according to claim 1, including apparatus that enables viewing where said selected object region is a planar region defined as a set of all points in the immediate vicinity of a plane that are in a selected portion of said volume.

9. An imaging system according to claim 1, including apparatus that operates to cause transmit-receive events for respective said spots includes operation where a plurality of transmit events overlap in time such that said focus spots rapidly cover a said selected object region.

10. An imaging system according to claim I, including apparatus to hold a coupling substance between an active surface of said transducer apparatus and said volume, such that said medium is extended to enable ultrasonic propagation between said transducer apparatus and said volume.

11. An imaging system according to claim 1, including apparatus to hold a coupling substance between an active surface of said transducer apparatus and said volume, such that said medium is extended to enable ultrasonic propagation between said transducer apparatus and said volume, and said coupling substance is a fluid that enables motion of said transducer apparatus relative to said volume.

12. An imaging system according to claim 1, including apparatus to hold a coupling substance between an active surface of said transducer apparatus and said volume, such that said medium is extended to enable ultrasonic propagation between said transducer apparatus and said volume, where said coupling substance is an attenuation leveling substance, where attenuation characteristics of said coupling substance match attenuation effects of that portion of said medium that is contained within said volume, such that said attenuation leveling substance enables propagation of wavefronts where relative amplitudes of respective signals that are distributed over a wavefront are not significantly changed by attenuation effects of said medium.

13. An imaging system according to claim 1, where said transducer apparatus is arranged such that transmitting transducers are also receiving transducers.

14. An imaging system according to claim 1, where said transducer apparatus is arranged such that transmitting transducers and receiving transducers are distinct from each other.

15. An imaging system according to claim 1, where said transducer apparatus is arranged as a first array and a second array that in a bistatic arrangement, where said first array and said second array are separated, and for a given said transmit-receive event, a transmit operation is enabled by said first array and a receive operation is enabled by said second array.

16. An imaging system according to claim 1, where said transducer apparatus is arranged as a first array and a second array in a bistatic arrangement, where said first array and said second array are separated, and for a given said transmit-receive event, a transmit operation is enabled by said first array and a receive operation is enabled by said second array, and transmit wavefronts produced by said transmit operation are substantially separate from that portion of wavefronts that develops into a wavefront portion that is received by said receive operation, where such separation suppresses cross-over interference signals.

17. An imaging system according to claim 1, and mechanical apparatus that provides mechanical motion of said transducer apparatus such that a plurality of said selected spots are positioned by operation that includes operation of said mechanical apparatus.

18. An imaging system according to claim I that is adapted for breast imaging, where said volume is a volume of breast tissue.

19. An imaging system that is adapted to image objects in a selected object region in a volume, where said volume includes a medium of propagation that enables ultrasonic wave propagation in said object region, where objects in said selected object region cause ultrasonic effects, where said imaging system includes
transducer apparatus that transmits and receives waves that propagate in said medium, where said transducer apparatus enables spot focusing of propagating waves at focus points in said selected object region, where said spot focusing enables approximately exclusive sensing in selected spots, where a spot is a volume region about a focus point that is enclosed by diffraction limited effects that result from focusing of a propagating wave, where a coupling fluid is contained between said transducer apparatus and said volume to enable propagation of ultrasonic waves between an active transducer surface and said volume, and containment of said coupling fluid is arranged to enable motion of said transducer apparatus relative to said volume, and said coupling fluid has attenuation characteristics at system operating frequencies that match respective attenuation characteristics at said system operating frequencies of said medium of propagation, and apparatus that operates to cause transmit-receive events for respective said selected spots, where a transmit-receive event includes transmitting of signals to form a focused outgoing wave that is focused for a selected spot and receiving of signals to cause focusing, for said selected spot, of incoming waves, where operation of said transmit-receive event enables selective sensing of ultrasonic effects that occur in said selected spot, where said transmit-receive events cause controlled focus point positions such that said selected spots approximately continuously cover said selected object region in said volume, and
apparatus that operates to convert results of said selective sensing to enable display of an image that enables viewing of said objects.

20. An imaging system according to claim 19, and correlation apparatus to correlate signals in a pre-set time relationship, where a correlation channel is set for an arrival time of an assumed signal from said selected spot, such that a replica signal and said assumed signal would correlate to produce a data sample that would indicate amplitude of said assumed signal, where operation of said correlation apparatus thus produces data samples that enable determination of image signals.

21. An imaging system according to claim 19, where said transducer apparatus is a cylindrical transducer apparatus having a cylindrical axis, where said cylindrical transducer has an active surface that enables focusing of ultrasonic waves at focus points that are distributed along said cylindrical axis.

22. An imaging system according to claim 19, where said transducer apparatus is a cylindrical transducer apparatus having a cylindrical axis, where said cylindrical transducer has an active surface that enables focusing of ultrasonic waves at focus points that are distributed along said cylindrical axis, where said cylindrical transducer apparatus includes a cylindrical transmitting transducer apparatus and a cylindrical receiving transducer apparatus that are separate.

23. An imaging system according to claim 19, where said motion includes a mechanical scanning motion of said transducer apparatus that enables scanning over a said selected object region that is a region about a plane.

24. An imaging system according to claim 19, where said transducer apparatus includes two arrays of transducers that are sparse arrays, where a first array has a first sparse element distribution that causes grating lobes in a first set of positions, and a second array has a second sparse element distribution that causes grating lobes in a second set of positions, and said first set of positions does not intersect with said second set of positions.

25. An imaging system according to claim 19, and apparatus to cause hybrid beamforming, where an active aperture is shifted by changing signal path connections to cause shift in a lateral direction of a focus spot, where said switching causes a coarse increment change in position of said focus spot, and signal processing means to cause an angle adjustment of focused wave propagation, to cause a fine increment change in position of said focus spot, and a hybrid effect of coarse increment changes and fine increment changes enables scanning that causes continuous focus spots in said lateral direction.

26. An imaging system according to claim 19, and apparatus to compensate for attenuation variations as a function of frequency, where said coupling fluid enables propagation where all waves at each frequency are subjected to an approximately known attenuation rate per unit distance, where compensation requires adjusting each frequency component of signals to cause compensated signals, where frequency components of a compensated signal have a constant amplitude over said operating frequencies, where determination of adjustments for a spot is enabled by said approximately known attenuation rate per unit distance.

27. An imaging system according to claim 19, where said controlled focus point positions are controlled by a combination of mechanical and electronic apparatus.

28. A large aperture array of transducers configured to enable ultrasonic focusing at a focus spot in a propagation medium, where said transducers are formed as strips that are attached to a backing surface, and said strips are of transducer material that enables a transducer function that couples ultrasonic operation with electrical operation, where said electrical operation is in relation to an electric field that is parallel to said backing surface.

29. An array according to claim 28, where said strips are cut into segments that cause a strip to function as a plurality of individual transducers that are connected to respective electric circuits.
